(19) United States
(12) Patent Application Publication
(10) Pub. No.: US 2023/0105077 A1
(43) Pub. Date: Apr. 6, 2023

(54) METHOD AND SYSTEM FOR EVALUATING AND MONITORING COMPLIANCE, INTERACTIVE AND ADAPTIVE LEARNING, AND NEUROCOGNITIVE DISORDER DIAGNOSIS USING PUPILLARY RESPONSE, FACE TRACKING EMOTION DETECTION

(71) Applicant: Yuen Lee Viola Lam, Hong Kong (HK)

(72) Inventors: Johan Matthijs DOLSMA, Hong Kong (HK); Yuen Lee Viola LAM, Hong Kong (HK)

(21) Appl. No.: 17/960,835

(22) Filed: Oct. 6, 2022

Related U.S. Application Data
(63) Continuation-in-part of application No. 16/313,895, filed on Dec. 28, 2018, now Pat. No. 11,475,788.
Application No. PCT/IB2018/053968 is a continuation-in-part of application No. 15/647,272, filed on Jul. 12, 2017, now abandoned.
(60) Provisional application No. 62/520,542, filed on Jun. 15, 2017.
(60) Provisional application No. 62/594,557, filed on Dec. 5, 2017.
(60) Provisional application No. 62/622,888, filed on Jan. 27, 2018.
(60) Provisional application No. 62/627,734, filed on Feb. 7, 2018.
(60) Provisional application No. 62/646,365, filed on Mar. 21, 2018.

(51) Int. Cl. G06F 16/22; G06N 20/00

(52) U.S. Cl. CPC G06F 16/2246; G06N 20/00

(57) ABSTRACT

A system for administering, evaluating, and monitoring a subject’s compliance with task performance requirements within an action programme, or for detection of noncompliance including substance abuse, driving under influence, and untruthful testimony giving under law enforcement setting, comprising optical sensors for capturing subject’s pupillary responses, blinking eye movements, eye movements, point-of-gaze, head pose, and facial expression of a subject. The system can also be applied in neurocognitive disorder diagnosis. The subject’s affective and cognitive states estimation based on the captured sensory data during a diagnosis test is feedback to the system to drive the course of the compliance or cognitive test, adaptively change the test materials, and influence the subject’s affective and cognitive states. The estimated affective and cognitive states in turn provide a more accurate reading of the subject’s condition.

### CROSS-REFERENCES WITH RELATED DOCUMENTS

This application is a continuation-in-part application of U.S. Pat. Application No. 16/313,895 filed on Dec. 28, 2018, the disclosure of which are incorporated herein by reference in its entirety.

### FIELD OF THE INVENTION

The present invention relates generally to methods and systems for providing and delivery of compliance administration and monitoring in the contexts of educational programmes and training, including corporate training, academic tutoring, in-class and out-of-class learnings, medical treatment and health improvement programmes, sport training, fitness and lifestyle programmes, correctional service and rehabilitation programmes, as well as governmental law and regulatory enforcement, and behavioral standards for individuals; interactive and adaptive learning; neurocognitive disorder diagnosis, and electronic commerce and retailing. Particularly, the present invention relates to to the techniques and applications of pupillary response detection, face tracking and emotion detection and analysis in aforementioned methods and systems.

### BACKGROUND OF THE INVENTION

Compliance means conforming to certain task performance specifications as required by an action programme by a subject enrolled therein. Conventional compliance evaluation and monitoring techniques focus on providing one-time testing with clearly defined passing criteria to subjects in an action programme. Many of these conventional techniques focus only on obtaining pass/fail indicators as effectively as possible without any capability for predicting the subject’s progress or guiding the subject towards total compliance. Further, these traditional compliance evaluation and monitoring methods often rely on one-time or sparse, at best, manual administrations of tests. This can hardly provide assurance for compliance on a continuous basis.

Law enforcements nowadays are facing increasingly complex cases everyday yet are expected by the public to make instant judgements, and rapid, appropriate and justifiable actions. Being able to systematically and accurately estimate the subject’s affective and cognitive states in real-time can aid in cases such as detecting substance abuse in suspects, driving under influence (DUI), unreliable witnesses, and untruthful testimony.

Part of the delivery of education services is the diagnosis and understanding of psychological disorders such as autism spectrum and attention deficit hyperactivity disorder (ADHD), which has received much attention lately. Educators are expected to spot these disorders in the students, but diagnosing these disorders is difficult and requires professional judgement by the subject matter experts.

Regarding psychological disorders early diagnosis, as the populations of most developed countries are all getting older, medical and elderly care systems are increasingly stretched for resources and care providers. One of the areas of medical and elderly care that often receives the least attention is the neurocognitive disorder diagnosis, prevention, and treatment. Even though early and accurate diagnosis of the various types of neurocognitive disorders can lead to effective treatments, similar to the academic and corporate training settings, accessibility to qualified professionals is an issue. Similarly, there is an unmet need for a systematic approach to the diagnosis of neurocognitive disorders by monitoring the subject’s affective and cognitive states, processing and analyzing the gathered data for making accurate diagnosis.

To address aforementioned issues, it would be desirable to have an intelligent compliance evaluation and monitoring system that models the affective and cognitive states of the subject, assist the compliance officer, law enforcement officer, teacher/trainer, or medical service provider in providing personalized compliance instructions and questionnaire in guiding the subject toward total compliance, learning/training programme, or medical treatment, continuously monitoring the subject’s task performance and behavior, and minimize overhead activities.

### SUMMARY OF THE INVENTION

The present invention provides a method and system for administering, evaluating, and monitoring a subject’s compliance with task performance requirements within an action programme using one or more of sensing of the subject’s pupillary responses, eye movements, gestures, emotions, and movements, speech and voice recognition, behavior pattern recognition, quantitative measurements of questionnaire results and task performances, and combinations thereof.

It is also an objective of the present invention to provide such method and system that are adaptable to workplace performance monitoring and appraisal assessment. It is still another objective of the present invention to provide such method and system adaptable to providing neurocognitive disorder diagnosis in education and training contexts, general medical services environment, and elderly care environment. It is still another objective of the present invention to provide such method and system adaptable to law enforcement settings in which the detection of noncompliance such as substance abuse in suspects, DUI, unreliable witnesses, and untruthful testimony relies on the combination of the subject’s physiological responses including pupillary responses, eye movements, gestures, body movements, emotions, and quantitative measurements of test assessments.

In accordance to various embodiments of the present invention, the subject within the action programme is to perform certain tasks according to performance specifications. In accordance to one embodiment, the method and system evaluate and monitor the compliance of the subject by periodically administering one or more questionnaires and analyzing the subject’s responses to the questionnaires; and by continuously monitoring the subject’s performance of one or more tasks under performance specification requirements.

In accordance to one aspect of the present invention, the method and system estimates the affective state and cognitive state of the subject by image and/or video capturing and analyzing the subject’s iris, or more specifically pupils, of one or both eyes, eye movements, blinking, point-of-gaze, facial expression, and head pose; and physiologic detection, such as tactile pressure exerted on a tactile sensing device, subject’s handwriting, tone of voice, and speech clarity during the time when the subject is responding to the questionnaire or during a sampling time window when the subject is performing the task procedure. The image or video capture can be accomplished by using built-in or peripheral cameras in desktop computers, laptop computers, tablet computers, and/or smartphones used by the subject in responding to the questionnaire, and/or other optical sensing devices placed and installed in the environments within which the subject performs the tasks in the action programme. The captured images and/or videos are then analyzed using machine vision techniques. For example, stalled eye movements, out-of-focus point-of-gaze, and a tilted head pose are signals indicating lack of interest and attention toward, and/or lack of knowledge in the subject matters being presented in the questionnaire and task procedural instructions, untruthfulness in answering the questionnaire, or lack of skill/knowledge in the tasks at hands; while a strong tactile pressure detected is a signal indicating anxiety, lack of confidence, and/or frustration in the subject matters being presented in the questionnaire and task procedural instructions or of the tasks at hands; either could represent a tendency of low level of compliance or noncompliance. For another example, pupil dilation detected may indicate dishonesty, uncertainty, anxiety, fight-or-flight emotion.

In accordance to one embodiment, selected performance data and behavioral data from the subject are also collected in determining the subject’s comprehension of and level of engagement in the materials presented in the questionnaires and task procedural instructions for tasks required to be performed in the action programme. These selected performance data and behavioral data include, but not limited to, correctness of answers to questions in the questionnaire, number of successful and unsuccessful attempts, closeness of the subject’s answers to model answers, number of toggling between given answer choices, and response speed to questions of certain types, and subject matters. For example, the subject’s excessive toggling between given choices and slow response speed in answering a question indicate doubts and hesitations on the answer to the question.

The affective state and cognitive state estimation and performance data are primarily used in gauging the subject’s level of compliance with performance specifications of tasks in an action programme. While a single estimation is used in providing a snapshot assessment of the subject’s progress toward total compliance in her task performance and prediction of the subject’s eventual achievable level of compliance, multiple estimations are used in providing an assessment history and trends of the subject’s progress. Furthermore, the estimated affective states and cognitive states of the subject are used in the modeling of the compliance programme in terms of choice of methods of compliance evaluation and monitoring, and instruction delivery and administration.

In accordance to another aspect of the present invention, the method and system provide a mechanism for delivering and managing interactive and adaptive compliance questionnaire and task procedural instructions. The mechanism logically structures the questionnaire and task procedural instructions materials and the delivery mechanism data for evaluating and monitoring compliance in an action programme as Domain Knowledge, and its data are stored in a Domain Knowledge repository. A Domain Knowledge repository comprises one or more Concept objects and one or more Task objects. Each Concept object comprises one or more Knowledge and Skill items. The Knowledge and Skill items are ordered by task performance specification complexity/difficulty/stringency levels, and two or more Knowledge and Skill items can be linked to form a Curriculum. In the case where the present invention is applied in a particular industry or business, a Curriculum defined by the present invention may be the equivalence of the operation manual/standard and there is one-to-one relationship between a Knowledge and Skill item and a task performance specification in the operation manual/standard. The Concept objects can be linked to form a logical tree data structure for used in a Task selection process.

Each Task object has various task procedural instruction materials. Each Task object is associated with one or more Concept objects in a Curriculum. In accordance to one embodiment, a Task object can be classified as: Basic Task, Interactive Task, or Task with an Underlying Cognitive or Expert Model. Each Basic Task comprises one or more operation notes, task procedural instructions, illustrations, test questions and answers designed to assess whether the subject has read all the materials. Each Interactive Task with an Underlying Cognitive or Expert Model comprises one or more task procedures each comprises one or more instructional steps designed to guide the subject in completing the task procedure according to performance specification. Each step provides an answer, common misconceptions, and hints. The steps are in the order designed to follow the delivery flow of a task procedure. This allows a tailored scaffolding (e.g., providing guidance and/or hints) for each task based on a point in a task procedure executed.

In accordance to another aspect of the present invention, the mechanism for delivering and managing interactive and adaptive compliance questionnaires and instructions logically builds on top of the Domain Knowledge two models of operation: Subject Model and Training Model. Under the Subject Model, the system executes each of one or more of the Task objects associated with a Curriculum in a Domain Knowledge in a work session for a subject. During the execution of the Task objects, the system measures the subject’s performance and obtain the subject’s performance metrics in each Task such as: the numbers of successful and unsuccessful attempts to complete the instructional steps in the Task, number of hints requested, and the time spent in completing the Task The performance metrics obtained, along with the information of the Task object, such as its specification complexity/difficulty/stringency level, are fed into a logistic regression mathematical model of each Concept object associated with the Task object. This is also called the knowledge trace of the subject, which is the calculation of a probability of the subject achieving a target compliance level in a task associated with the with the task performance specification in the Concept object. The advantages of the Subject Model include that the execution of the Task objects can adapt to the changing ability of the subject. For non-limiting example, following the Subject Model, the system can estimate the compliance level achievable by the subject, estimate how much performance improvement can be expected for a next Task, and provide a prediction of the subject’s level of compliance in a future point of time. These data are then used in the Training Model and enable hypothesis testing to make further improvement to the system, evaluate compliance officer quality and compliance questionnaire and task procedural instruction material quality.

Under the Training Model, the system receives the data collected from the execution of the Task objects under the Subject Model and the Domain Knowledge for making decisions on the instruction delivery strategy and providing feedbacks to the subject and compliance officer. Under the Training Model, the system is mainly responsible for executing the followings:
1.) Define the entry point for the first Task. Initially all indicators for Knowledge and Skill items are set to defaults, which are inferred from data in either an application form filled by the subject or compliance officer or an initial assessment of the subject by the compliance officer. Select the sequence of Tasks to execute. To select the next Task, the system’s trainer module has to search through a logical tree data structure of Concept objects, locate a Knowledge and Skill with the lowest skill level and then use a question matrix to lookup the corresponding Task items that match the learning traits of the subject. Once selected, the necessary compliance questionnaire and task procedural instruction materials are pulled from the Domain Knowledge, and send to the system’s communication module for delivery presentation in the system’s communication module user interface.
2.) Provide feedback. While the subject is working on a Task object being executed, the system’s trainer module monitors the time spent on each Task step. When a limit is exceeded, feedback is provided as a function of the current affective state of the subject. For example, this can be an encouraging, empathetic, or challenging message selected from a generic list, or it is a dedicated hint from the Domain Knowledge.
3.) Drive the system’s pedagogical agent. The system’s trainer module matches the current affective state of the subject with the available states in the pedagogical agent. Besides providing the affective state information, text messages can be sent to the system’s communication module for rendering the pedagogical agent in a user interface.
4.) Decide when a Concept is mastered. As described earlier, under the Subject Model, the system estimates the probability of the subject achieving a target compliance level in a task associated with the task performance specification materials in each Concept. Based on a predetermined threshold (e.g., 95%), the compliance officer can decide when a Concept is mastered.
5.) Flag subject’s behavior that is recognized to be related to mental disorders. For example, when the system’s execution under the Subject Model shows anomalies in the sensor data compared to a known historical context and exhibits significant lower learning progress, the system under the Training Model raises a warning notice to the teacher/trainer. It also provides more detailed information on common markers of disorders such as Attention Deficit Hyperactivity Disorder (ADHD) and Autism Spectrum Disorder (ASD).

In accordance to another aspect of the present invention, the method and system for administering, evaluating, and monitoring a subject’s compliance with task performance requirements within an action programme incorporate machine learning techniques that are based on interlinking models of execution comprising: a Domain Model, an Assessment Model, a Learner Model, a Deep Learner Model, one or more Motivational Models, a Transition Model, and a Pedagogical Model. The interlinking models of execution is purposed for driving, inducing, or motivating certain desirable actions, behavior, and/or outcome from the subject. These certain desirable actions and/or outcome can be, as non-limiting examples, mastering certain task procedures, adopting certain desirable behaviors, achieving certain job assignment goals, making certain purchases, and conducting certain commercial activities. Therefore, these interlinking models of execution are also applicable in the fields of education, corporate training and commercial retailing and trading.

The present invention can also be applied in medical assessment for cognitive disorders, such as Alzheimers’ dementia and autism ADHD. In accordance to one embodiment, provided is a neurocognitive disorder diagnosis system for administering a cognitive test comprising questionnaire and task procedural instruction materials administered to a subject. The system monitors and estimates the subject’s affective state and cognitive state using the collected and analyzed sensory data on subject’s iris, or more specifically pupils, of one or both eyes, eye movements, blinking, point-of-gaze, facial expression, head pose, voice, speech clarity, reaction time, and/or touch responses during the cognitive test. This is similar to the aforesaid methods and systems for evaluating and monitoring compliance.

The cognitive test may be delivered by a human test administrator in the presence of the subject, remotely through electronic means (e.g., network connected personal computers and tablet computers, and smartphone), or automatically through a specially configured computing device. The cognitive test materials can be a series of textual, pictorial, and/or video based questions based on the subject’s knowledge on distanced events and recent events so to assess the patient subject’s states of long-term memory and short-term memory respectively through memory recall time and accuracy as part of the patient subject’s test performance data. The subject’s affective state and cognitive state estimation, along with the patient subject’s cognitive test performance data during the cognitive test are feedback to the system to drive the course of the cognitive test, to adaptively change the cognitive test materials, and in turn to influence the subject’s affective and cognitive state in a closed loop feedback system.

The neurocognitive disorder diagnosis system provides a real-time diagnosis that is less prone to human error. The subject’s affective state and cognitive state estimation can also be matched and used alongside with MRI data on the subject’s brain activity in further study.

The goal of neurocognitive disorder diagnosis system is to enable the early detection of cognitive disorders, particularly among elderly in elderly care facilities such as retirement homes, through the periodic administrations of cognitive tests using this system. Another goal is to enable the tracking of treatments, and in turn drive the adjustments in the course of the treatments, medications, and frequencies of doctor’s visits.

Embodiments of the present invention can also be adapted to law enforcement applications, including real-time detection of substance abuse in suspects, DUI, unreliable witnesses, and untruthful testimony. The detection of substance abuse or DUI comprises a delivery of a test which can be a series of textual, pictorial, and/or video based contextual questions, light stimulus (to the subject’s eyes), and/or physical task performance (e.g., handwriting, passage reading, repeating directed hand gesture, and walking with directions). Built-in or peripheral cameras in desktop computers, laptop computers, tablet computers, and/or smartphones, standalone cameras, and/or infrared cameras (under low light conditions) can be used to track and image and/or video-capture the subject’s iris, or more specifically pupils, of one or both eyes, eye movements, blinking, point-of-gaze, facial expression, and head pose. Other physiological actions, such as the subject’s handwriting, tone of voice, and speech clarity during the subject is responding to a written or verbal questionnaire; and physical movements within a geographical area can similarly be tracked and captured. The captured images and/or videos are then analyzed using machine vision techniques. For example, stalled eye movements, out-of-focus point-of-gaze, and pupil dilation detected during a test for substance abuse or DUI may indicate intoxication. For another example, frequent blinking and pupil dilation detected during interrogation may indicate lies telling by the suspect or witness. The combination of multiple different types of physiological response detected together with the estimation of affective and cognitive states of the subject using these physiological responses provide higher accuracy in the estimation of the condition of the subject.

### BRIEF DESCRIPTION OF THE DRAWINGS

Embodiments of the invention are described in more detail hereinafter with reference to the drawings, in which:

FIG. 1 depicts a schematic diagram of a system for administering, evaluating, and monitoring a subject’s compliance with task performance requirements within an action programme in accordance with one embodiment of the present invention;

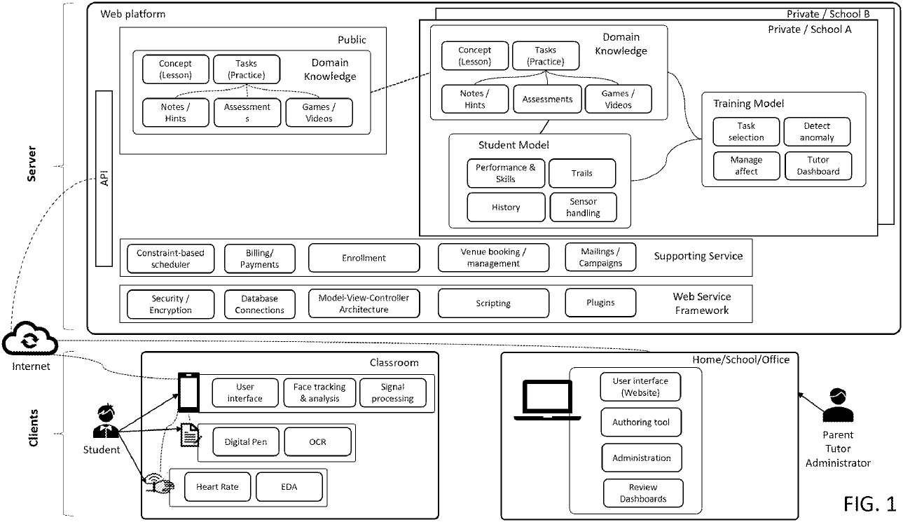

FIG. 2A depicts a logical data flow diagram of the system for administering, evaluating, and monitoring a subject’s compliance in accordance with one embodiment of the present invention;

FIG. 2B depicts a logical data flow diagram of the system for delivering and managing interactive and adaptive learning and training programmes in accordance with one embodiment of the present invention;

FIG. 3 depicts an activity diagram of a method for administering, evaluating, and monitoring a subject’s compliance with task performance requirements within an action programme or delivering and managing interactive and adaptive learning and training programmes in accordance with one embodiment of the present invention;

FIG. 4 depicts a flow diagram of an iterative machine learning workflow used by the system in calculating a probability of achieving a target compliance level by the subject;

FIG. 5 illustrates a logical data structure used by the system in accordance with one embodiment of the present invention;

FIG. 6A depicts a logical block diagram of interlinking models of execution in accordance with one aspect of the present invention; and

FIG. 6B shows the logical components details of the interlinking models of execution.

### DETAILED DESCRIPTION_{:}

In the following description, methods and systems for administering, evaluating, and monitoring a subject’s compliance, delivering and managing interactive learning and training programmes, neurocognitive disorder diagnosis with task performance requirements within an action programme and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance to various embodiments of the present invention, the method and system for administering, evaluating, and monitoring a subject’s compliance with task performance requirements within an action programme use a combination of sensing of the subject’s pupillary responses, eye movements, gestures, emotions, and movements, speech and voice recognition, behavior pattern recognition, quantitative measurements of questionnaire results and task performances, and combinations thereof.

In accordance to one aspect of the present invention, the method and system estimate the affective state and cognitive state of the subject by image and/or video capturing and analyzing the subject’s iris, or more specifically pupils, of one or both eyes, eye movements, blinking, point-of-gaze, facial expression, head pose, and haptic feedback, such as tactile pressure exerted on a tactile sensing, subject’s handwriting, tone of voice, and speech clarity during the time when the subject is responding to the questionnaire or during a sampling time window when the subject is performing the task procedure.

The image or video capture can be performed by using built-in or peripheral cameras in desktop computers, laptop computers, tablet computers, and/or smartphones used by the subject, and/or other optical sensing devices placed and installed in the environments within which the subject performs the tasks in the action programme. The captured images and/or videos are then analyzed using machine vision techniques. For example, stalled eye movements, out-of-focus point-of-gaze, and a tilted head pose are signals indicating lack of interest and attention, and/or lack of knowledge in the subject matters being presented in questionnaire or lecture materials, untruthfulness in answering the questionnaire or lecture materials, or lack of skill / knowledge in the tasks at hands or lecture materials; while a strong tactile pressure detected is a signal indicating anxiety, lack of confidence, and/or frustration in the subject matters being presented in questionnaire or of the tasks at hands or of the lecture materials; either could represent a tendency of low level of compliance or noncompliance.

For another example, pupil dilation detected may indicate dishonesty, uncertainty, anxiety, fight-or-flight emotion. It has been known in the art that pupillary responses have physiological correlations to cognitive activities as disclosed in Kahneman, D., Attention and Effort, Prentice-Hall, U.S.A. (1973); the disclosure of which is incorporated herein by reference in its entirety. Yet for another example, a strong tactile pressure detected is a signal indicating anxiety, lack of confidence, and/or frustration in the subject matters being presented in in questionnaire or of the tasks at hands or of the lecture materials.

In accordance to one embodiment, selected performance data and behavioral data from the subject are also collected in the affective state and cognitive state estimation. These selected performance data and behavioral data include, but not limited to, number of successful and unsuccessful attempts to task procedural step completions, speed in completing task procedures, correctness of answers to questions in the questionnaire, number of successful and unsuccessful attempts to questions, closeness of the subject’s answers to model answers, toggling between given answer choices, and response speed to test questions of certain types, subject matters, and/or task performance specification complexity/difficulty/stringency levels, working steps toward a solution, the subject’s handwriting, tone of voice, and speech clarity. For example, the subject’s excessive toggling between given choices and slow response speed in answering a test question indicating doubts and hesitations on the answer to the question. The subject’s intermediate working steps toward completing a task procedural step are captured for matching with the model solution and in turn provides insight to the subject’s understanding of the task procedural instruction and task performance specification or lecture materials.

In accordance to various embodiments, the system for administering, evaluating, and monitoring a subject’s compliance, interactive learning and training programmes, and neurocognitive disorder diagnosis with task performance requirements within an action programme comprises a sensor handling module implemented by a combination of software and firmware executed in general purposed and specially designed computer processors. The sensor handling module manages the various sensors employed by the system. The sensor handling module is in electrical and/or data communications with various electronic sensing devices including, but not limited to, optical and touch sensing devices; input devices including, but not limited to, keyboard, mouse, pointing device, stylus, and electronic pen; image capturing devices; and cameras.

During the operation of the system, input sensory data are continuously collected at various sampling rates and averages of samples of input sensory data are computed. In order to handle the different sampling rates of different sensing devices, a reference rate is chosen (e.g., 5 Hz). A slower sampling rate input sensory data is interpolated with zero order hold and then sampled at the reference rate. A higher sampling rate input sensory data is subsampled at the reference rate. After the sample rate alignment, a trace of the last few seconds is kept in memory after which the average is calculated. Effectively this produces a moving average of an input sensory data and acts as a lowpass filter to remove noise.

### Pupillary Responses, Blinking, Eye Movements, Point-of-gaze, and Head Pose Detection

In one embodiment, one or more low-cost optical sensor built-in in a computing device (e.g., subject facing camera in a tablet computer) is used to image and/or video-capture a subject’s eyes and face. At a rate of minimal 5 Hz, images are obtained from the sensor. A sampling rate of at least 60 Hz is recommended for capturing pupil changes, eye blinking, and subtle eye movements. Each image is then processed by face/eye tracking and analysis systems known in the art. The three-dimensional (3D) head orientation is measured in Euler angles (pitch, yaw, and roll). First, to measure the point-of-gaze, a 3D vector is assumed from the origin of the optical sensor to the center of the pupil of the subject. Secondly, a 3D vector is determined from the center of the eye-ball to the pupil. These two vectors are then used to calculate the point of gaze. A calibration step helps to compensate for offsets (subject position behind the screen, camera position relative to the screen). Using this data, the planar coordinate of the gaze on the computer screen can be derived. The time duration of a fixed point of gaze, changes in point of gaze, and changes in head pose can be obtained from multiple images and/or video frames captured. Pupil diameter changes of the two eyes can also be extracted from multiple images and/or video frames captured. Similarly, blinking frequency can be calculated from the number of the images and/or video frames captured showing closed eyes during a defined period of time.

### Facial Expressions and Emotions Determination

In another embodiment, the images and/or videos captured as mentioned above, are processed to identify key landmarks on the face such as eyes, tip of the nose, corners of the mouth. The regions between these landmarks are then analyzed and classified into facial expressions such as: attention, brow furrow, brow raise, cheek raise, chin raise, dimpler (lip corners tightened and pulled inwards), eye closure, eye widen, inner brow raise, jaw drop, lid tighten, lip corner depression, lip press, lip pucker (pushed forward), lip stretch, lip such, mouth open, nose wrinkle, smile, smirk, upper lip raise. These expressions are then mapped, using a lookup table, onto the following emotions: anger, contempt, disgust, engagement (expressiveness), fear, joy, sadness, surprise and valence (both positive as negative nature of the person’s experience). Each emotion is encoded as a percentage and output simultaneously.

### Physiologic Measurement

In another embodiment, the system comprises a wearable device to measure physiologic parameters not limiting to: heart rate, electro dermal activity (EDA) and skin temperature. This device is linked wirelessly to the client computing device (e.g. tablet computer or laptop computer). The heart rate is derived from observations of the blood volume pulse. The EDA measures skin conductivity as an indicator for sympathetic nervous system arousal. Based on this, features related to stress, engagement, and excitement can be derived. Another approach is to use vision analysis techniques to directly measure the heart rate based on the captured images. This method is based on small changes in light absorption by the veins in the face, when the amount of blood varies due to the heart rate.

### Handwriting Analysis

In another embodiment, test answers may be written on a dedicated note paper using a digital pen and receive commands such as ‘task procedural step completed’. The written answer is then digitized on the fly and via an intelligent optical character recognition engine, the system can evaluate the content written by the subject and provide any necessary feedback to guide the subject when needed. Studies show that taking longhand notes encourages subjects to process and reframe information, improving the compliance or understanding of lecture materials. Alternatively, embodiments may use OCR after the tasks has been completed. The paper is scanned using a copier and the digitized image is fed to OCR software.

### Speech and Voice Recognition and Analysis

In another embodiment, the system comprises one or more voice recording devices for recording the subject’s speech during a compliance evaluation and monitoring session or learning/training session or neurocognitive disorder diagnosis. The subject’s speech is then digitized on the fly and via an intelligent voice recognition engine, the system can evaluate the content spoken by the subject and provide any necessary feedback to guide the subject when needed. The substantive content of the subject’s speech is recognized for verbal commands related to a task procedure and/or verbal answers to questionnaire test questions for further compliance, leaming/training, or neurocognitive analysis. The subject’s voice and speech clarity are recognized as input to the affective state and cognitive state estimation.

### Pedagogical Agent-subject Interaction

As a non-limiting example, a pedagogical agent may be non-human animated character with human traits implemented by a combination of software and/or firmware running in one or more general purposed computer processors and/or specially configured computer processors. It can display the basic emotions by selecting from a set of animations (e.g. animated GIFs), or by using scripted geometric transformation on a static image displayed to the subject in a user interface. Another method is to use SVG based animations. The animation can be annotated with text messages (e.g. displayed in a balloon next to the animation). The text messages are generated by and received from the trainer module of the system. The subject’s responses to the pedagogical agent are received by the system for estimating the subject’s affective state.

The affective state and cognitive state estimation and performance data are primarily used in gauging the subject’s level of compliance with performance specifications of tasks in an action programme or the subject’s understanding of and interests in the materials covered in a learning or training programme. While a single estimation is used in providing a snapshot assessment of the subject’s progress toward total compliance in her task performance or the learning or training programme and prediction of the subject’s eventual achievable level of compliance or the subject’s test results on the lecture materials, multiple estimations are used in providing an assessment history and trends of the subject’s progress. Furthermore, the estimated affective states and cognitive states of the subject are used in the modeling of the compliance programme in terms of choice of methods of compliance evaluation and monitoring, and instruction delivery and administration and of the learning or training programme in terms of choice of subject matter materials, delivery methods, and administration.

### Domain Knowledge

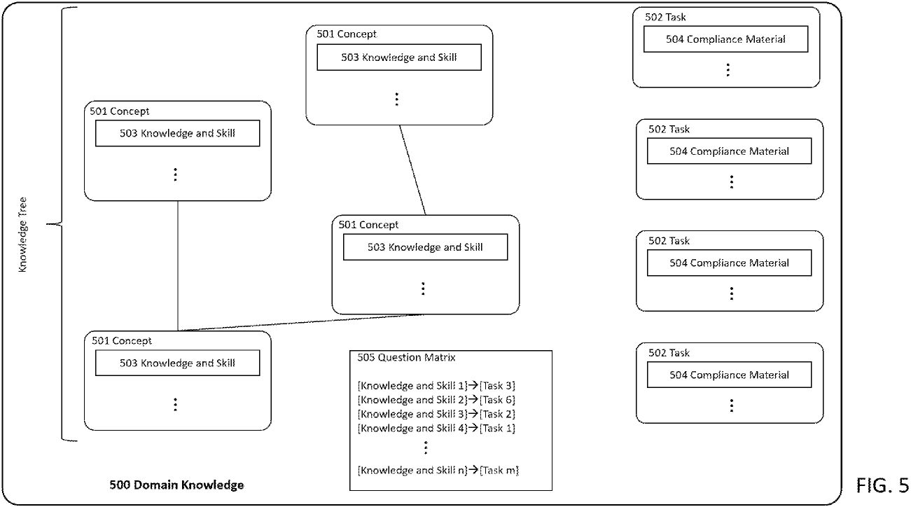

Referring to FIG. 5. In accordance to one aspect of the present invention, the method and system logically structure the compliance questionnaire and task procedural instruction materials, and the delivery mechanism in a compliance programme as Domain Knowledge 500. A Domain Knowledge 500 comprises one or more Concept objects 501 and one or more Task objects 502. Each Concept object 501 comprises one or more Knowledge and Skill items 503. The Knowledge and Skill items 503 are ordered by task performance specification complexity/difficulty/stringency levels, and two or more Concept objects 501 can be grouped to form a Curriculum. In the case where the present invention is applied in a particular industry or business, a Curriculum defined by the present invention may be the equivalence of the operation manual/standard and there is one-to-one relationship between a Knowledge and Skill item and a task performance specification in the operation manual/standard. The Concept objects can be linked to form a logical tree data structure (Knowledge Tree) such that Concept objects having Knowledge and Skill items that are fundamental and/or basic in a topic are represented by nodes closer to the root of the logical tree and Concept objects having Knowledge and Skill items that are more advance and branches of some common fundamental and/or basic Knowledge and Skill items are represented by nodes higher up in different branches of the logical tree.

Each Task object 502 has various compliance questionnaire and task procedural instruction content or lecture materials 504, and is associated with one or more Concept objects 501 in a Curriculum. The associations are recorded and can be looked up in a question matrix 505. In accordance to one embodiment, a Task object 502 can be classified as: Basic Task, Interactive Task, or Task with an Underlying Cognitive or Expert Model. Each Basic Task comprises one or more operation notes, task procedural instructions (e.g., video clips and other multi-media content), lecture notes, test questions and answers designed to assess whether the subject has read all the materials. Each Interactive Task with an Underlying Cognitive or Expert Model comprises one or more problem-solving exercises each comprises one or more steps designed to guide the subject in deriving the solutions to problems. Each step provides an answer, common misconceptions, and hints. The steps are in the order designed to follow the delivery flow of a task procedure or lecture. This allows a tailored scaffolding (e.g., providing guidance and/or hints) for each task based on a point in a task procedure or learning/training session or neurocognitive disorder diagnosis executed.

In accordance to various embodiments, a Task object gathers a set of compliance questionnaire and task procedural instruction, or lecture materials (e.g., operation notes and illustrations) relevant in the achievement of a compliance level. In addition to the aforementioned classification, a Task can be one of the following types:
1.) Reading Task: operation/lecture notes or illustrations to introduce a new topic without grading, required to be completed before proceeding to a Practice Task is allowed;
2.) Practice Task: a set of questions from one topic to practice on questions from a new topic until a threshold is reached (e.g., five consecutive successful attempts without hints, or achieve an understanding level of 60% or more);
3.) Mastery Challenge Task: selected questions from multiple topics to let the subject achieves mastery (achieve an understanding level of 95% or more) on a topic, and may include pauses to promote retention of knowledge (e.g., review opportunities for the subjects); or
4.) Group Task: a set of questions, problem sets, and/or problem-solving exercises designed for peer challenges to facilitate more engagement from multiple subjects in a focus group, maybe ungraded.

In accordance to one embodiment, the Domain Knowledge, its constituent Task objects and Concept objects, Knowledge and Skill items and Curriculums contained in each Concept object, operation notes, illustrations, test questions and answers in each Task object are data entities stored a relational database accessible by the system (a Domain Knowledge repository). One or more of Domain Knowledge repositories may reside in third-party systems accessible by the system for administering, evaluating, and monitoring a subject’s compliance with task performance requirements within an action programme.

In accordance to another aspect of the present invention, the mechanism for delivering and managing interactive and adaptive compliance questionnaires and task procedural instructions logically builds on top of the Domain Knowledge two models of operation: Subject Model and Training Model.

### Subject Model

Under the Subject Model, the system executes each of one or more of the Task objects associated with a Curriculum in a Domain Knowledge for a subject. During the execution of the Task objects, the system measures the subject’s performance and obtain the subject’s performance metrics in each Task such as: the numbers of successful and unsuccessful attempts to questions in the Task, number of hints requested, and the time spent in completing the Task. The performance metrics obtained, along with the information of the Task object, such as its specification complexity/difficulty/stringency level, are fed into a logistic regression mathematical model of each Concept object associated with the Task object. This is also called the knowledge trace of the subject, which is the calculation of the probability of the subject achieving a target compliance level in a task or understanding of the materials associated with the Concept object. In one embodiment, the calculation of a probability of achieving a target compliance level or level of understanding uses a time-based moving average of subject’s answer scores to questions in the questionnaires with lesser weight on older attempts, the number of successful attempts, number of failed attempts, success rate (successful attempts over total attempts), time spent, and task performance specification complexity/difficulty/stringency level. In another embodiment, the calculation of a probability of achieving a target compliance level or level of understanding uses a time-based moving average of subject’s completion of task procedural steps with lesser weight on older attempts, the number of successful attempts, number of failed attempts, success rate (successful attempts over total attempts), time spent, and task performance specification or question complexity/difficulty/stringency level.

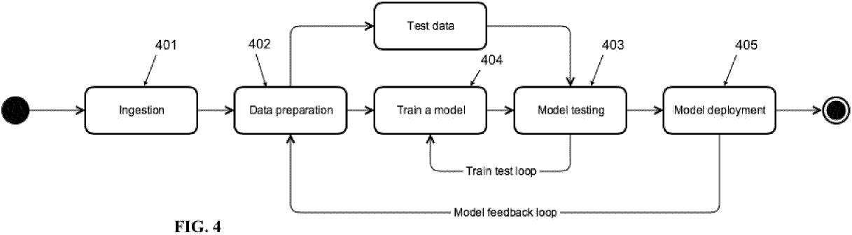

In one embodiment, the system calculates the probability of the subject achieving a target compliance level in a task associated with the task performance specification in the Concept object using an iterative machine learning workflow to fit mathematical models on to the collected data (subject’s performance metrics and information of the Task) including, but not limited to, a time-based moving average of subject’s answer scores to questions in the questionnaires with lesser weight on older attempts, the number of successful attempts, number of failed attempts, success rate (successful attempts over total attempts), time spent, topic difficulty, and question difficulty. FIG. 4 depicts a flow diagram of the aforesaid iterative machine learning workflow. In this exemplary embodiment, data is collected (401), validated and cleansed (402); then the validated and cleansed data is used in attempting to fit a mathematical model (403); the mathematical model is trained iteratively (404) in a loop until the validated and cleansed data fit the mathematical model; then the mathematical model is deployed (405) to obtain the probability of the subject achieving a target compliance level in a task or understanding of the materials associated with the task performance specification in the Concept object; the fitted mathematical model is also looped back to and used in the step of validating and cleansing of the collected data.

The knowledge trace of the subject is used by the system in driving Task compliance questionnaire and task procedural instruction or lecture material items selection, driving Task object selection, and driving compliance questionnaire and task procedural instruction or lecture material ranking. The advantages of the Subject Model include that the execution of the Task objects can adapt to the changing ability of the subject. For non-limiting example, under the Subject Model the system can estimate the compliance level or level of understanding achievable by the subject, estimate how much performance improvement can be expected for the next Task, and provide a prediction of the subject’s level of compliance in a future point of time. These data are then used in the Training Model and enable hypothesis testing to make further improvement to the system, evaluate compliance officer quality and compliance questionnaire and task procedural instruction or lecture material quality.

### Training Model

Under the Training Model, the system’s trainer module receives the data collected from the execution of the Task objects under the Subject Model and the Domain Knowledge for making decisions on the compliance questionnaire and task procedural instruction or lecture material delivery strategy and providing feedbacks to the subject and compliance officer or teacher/trainer or medical service provider. The system for administering, evaluating, and monitoring a subject’s compliance with task performance requirements within an action programme or delivering and managing interactive and adaptive learning and training programmes or administering neurocognitive disorder diagnosis comprises a trainer module implemented by a combination of software and firmware executed in general purposed and specially designed computer processors. In one embodiment, the trainer module resides in one or more server computers. The trainer module is primarily responsible for executing the machine instructions corresponding to the carrying-out of the activities under the Training Model. Under the Training Model, the trainer module executes the followings:
1.) Define the entry points for the Tasks execution. Initially all indicators for Concept Knowledge and Skill items are set to defaults, which are inferred from data in either an application form filled by the subject or compliance officer or teacher/trainer or medical service provider, or an initial assessment of the subject by the compliance officer or teacher/trainer or medical service provider. Select the subsequent Task to execute. To select the next Task, the system’s trainer module searches through a logical tree data structure of Concept objects (Knowledge Tree), locate a Concept Knowledge and Skill with the lowest skill level (closest to the root of the Knowledge Tree) and then use a matching matrix to lookup the corresponding Task object for making the selection. Once selected, the Task object data is retrieved from the Domain Knowledge repository, and send to the system’s communication module for delivery presentation.
2.) Provide feedback. While the subject is working on a Task object being executed, the system’s trainer module monitors the time spent on a Task step. When a time limit is exceeded, feedback is provided as a function of the current affective state of the subject. For example, this can be an encouraging, empathetic, or challenging message selected from a generic list, or it is a dedicated hint from the Domain Knowledge.
3.) Drive the system’s pedagogical agent. The system’s trainer module matches the current affective state of the subject with the available states in the pedagogical agent. Besides providing the affective state information, text messages can be sent to the system’s communication module for rendering along with the pedagogical agent’s action in a user interface displayed to the subject.
4.) Decide when a Concept is mastered. As described earlier, under the Subject Model, the system estimates the probability of the subject achieving a target compliance level in a task associated with the task performance specification materials in each Concept. Based on a predetermined threshold (e.g., 95%), the compliance officer or teacher/trainer or medical service provider can decide when a Concept is mastered.
5.) Flag subject’s behavior that is recognized to be related to mental disorders. For example, when the system’s execution under the Subject Model shows anomalies in the sensory data compared to a known historical context and exhibits significant lower learning progress, the system under the Training Model raises a warning notice to the compliance officer or teacher/trainer or medical service provider. It also provides more detailed information on common markers of disorders such as Attention Deficit Hyperactivity Disorder (ADHD) and Autism Spectrum Disorder (ASD).

In accordance to various embodiments, the system for administering, evaluating, and monitoring a subject’s compliance with task performance requirements within an action programme and/or delivering and managing interactive and adaptive learning and training programmes and/or administering neurocognitive disorder diagnosis further comprises a communication module implemented by a combination of software and firmware executed in general purposed and specially designed computer processors. In one embodiment, one part of the communication module resides and is executed in one or more server computers, and other part of the communication module resides and is executed in one or more client computers including, but not limited to, desktop computers, laptop computers, tablet computers, smartphones, and other mobile computing devices, among which some are dedicated for use by the subjects and others by compliance officer or teacher/trainer or medical service provider.

The communication module comprises one or more user interfaces designed to present relevant data from the Domain Knowledge and materials generated by the system operating under the Subject Model and Training Model to the subjects and the compliance officers. The user interfaces are further designed to facilitate user interactions in capturing user input (textual, gesture, image, and video inputs) and displaying feedback including textual hints and the simulated pedagogical agent’s actions. Another important feature of the communication module is to provide an on-screen (the screen of the computing device used by a subject) planar coordinates and size of a visual cue or focal point for the current Task object being executed. For a non-limiting example, when an operation note from a Task object is being displayed on screen, the communication module provides the planar coordinates and size of the operation note display area and this information is used to match with the collected data from a point-of-gaze tracking sensor in order to determine whether the subject is actually engaged in the Task (looking at the operation note).

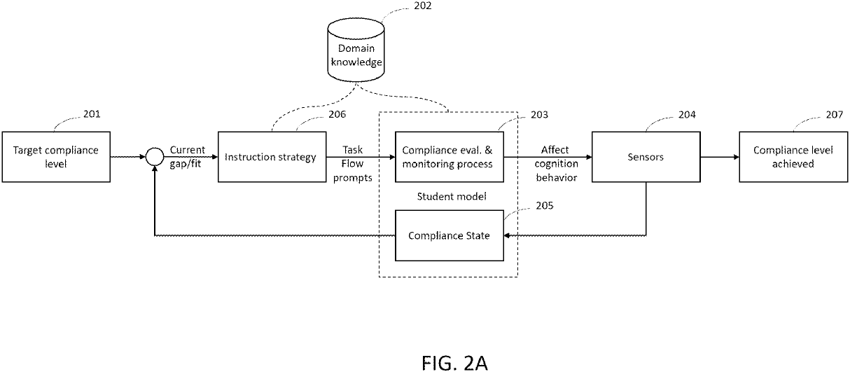

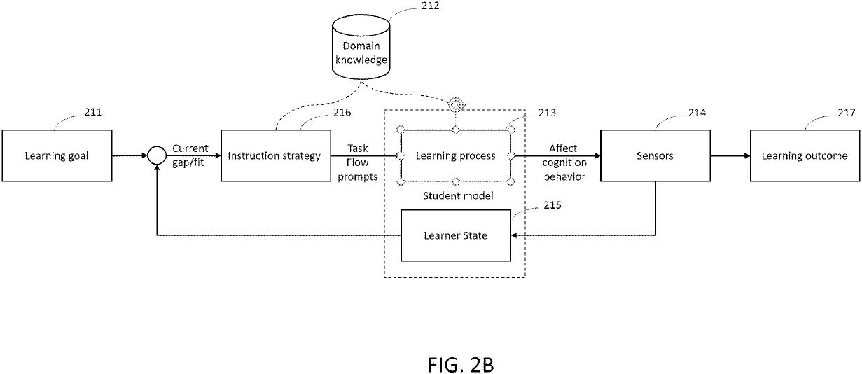

FIG. 2A depicts a logical data flow diagram of the system for administering, evaluating, and monitoring a subject’s compliance with task performance requirements within an action programme in accordance to various embodiments of the present invention. The logical data flow diagram illustrates how the major components of the system work together in a feedback loop in the execution during the Subject Model and Training Model. In an exemplary embodiment in reference to FIG. 2A, during enrollment, a suitable series of tasks is selected by the subject in an action programme. This series of tasks corresponds directly to a Curriculum object, which is a set of linked Concept objects in the Domain Knowledge 202, and constitutes the target compliance level 201 for this subject. Upon the subject logging into the system via a user interface rendered by the system’s communication module, under the Training Model, the system’s trainer module selects and retrieves from the Domain Knowledge 202 a suitable Concept object and the associated first Task object. Entering the Subject Model, the Task object data is retrieved from the Domain Knowledge repository, the system renders the Task object data (e.g. operation notes) on the user interface for the subject, and the subject starts working on the task. Meanwhile, the system manages the compliance evaluation and monitoring process 203 by collecting affective state sensory data including, but not limited to, point-of-gaze, emotion, and physiologic data, and cognition state data via Task questions and answers and the subject’s behavioral-analyzing interactions with the user interface (204). After analyzing the collected affective state sensory data and cognition state data, the compliance state 205 is updated. The updated compliance state 205 is compared with the target compliance level 201. The determined knowledge/skill gap or the fit of the task procedural instruction delivery strategy 206 is provided to the Training Model again, completing the loop. If the analysis on the collected affective state sensory data and cognition state data shows a probability of achieving certain compliance level that is higher than a threshold, that certain compliance level is considered achieved 207.

FIG. 2B depicts a logical data flow diagram of the system for delivering and managing interactive and adaptive learning and training programmes in accordance to various embodiments of the present invention. The logical data flow diagram illustrates how the major components of the system work together in a feedback loop in the execution during the Student Model and Training Model. In an exemplary embodiment in reference to FIG. 2B, during enrollment, a suitable course is selected by the student (or parents) in a learning or training programme. This course corresponds directly to a Curriculum object, which is a set of linked Concept objects in the Domain Knowledge 212, and constitutes the learning goal 211 for this student subject. Upon the student subject logging into the system via a user interface rendered by the system’s communication module, under the Training Model, the system’s trainer module selects and retrieves from the Domain Knowledge 212 a suitable Concept object and the associated first Task object. Entering the Student Model, the Task object data is retrieved from the Domain Knowledge repository, the system renders the Task object data (e.g., lecture notes, test questions, and problem set) on the user interface for the student subject, and the student subject starts working on the task. Meanwhile, the system monitors the learning process 213 by collecting affective state sensory data including, but not limited to, point-of-gaze, emotion, and physiologic data, and cognition state data via Task questions and answers and the student subject’s behavioral-analyzing interactions with the user interface (214). After analyzing the collected affective state sensory data and cognition state data, the learner state 215 is updated. The updated learner state 215 is compared with the learning goal 211. The determined knowledge/skill gap or the fit of the instruction strategy 216 is provided to the Training Model again, completing the loop. If the analysis on the collected affective state sensory data and cognition state data shows a probability of understanding that is higher than a threshold, the learning goal is considered achieved 217.

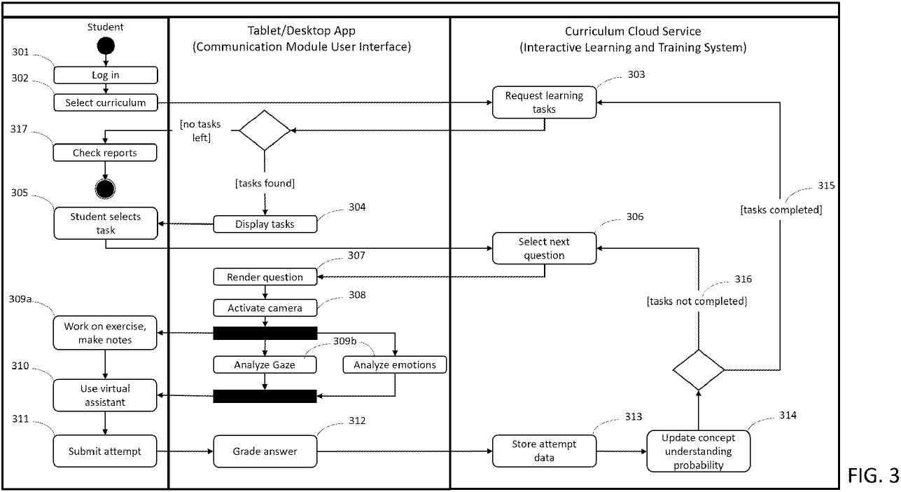

FIG. 3 depicts an activity diagram illustrating in more details the execution process of the system within an action programme under the Subject Model and Training Model. In an exemplary embodiment referring to FIG. 3, the execution process is as follows:
301. A subject logs into the system via her computing device running a user interface rendered by the system’s communication module.
302. The subject select a Curriculum presented to her in the user interface.
303. Upon receiving the user login, successful authentication, and receiving the Curriculum selection, the system’s trainer module, running in a server computer, selects and requests from the Domain Knowledge repository one or more Task objects associated with the Curriculum selected. When no Task object has yet been defined to associate with any Concept objects in the Curriculum selected, the system evaluates the Knowledge Tree and finds the Concept Knowledge and Skills that the subject has not yet learned and/or been evaluated as close to the root (fundamental) of the Knowledge Tree as possible. This process is executed by the system’s recommendation engine, which can be implemented by a combination of software and firmware executed in general purposed and specially designed computer processors. The recommendation engine can recommend Practice Tasks, and at lower rate Mastery Challenge Tasks. System-recommended Tasks have a default priority; compliance officer-assigned Tasks have a higher priority in the Task selection. In one embodiment, the system further comprises a recommendation engine for recommending the task performance specification materials (e.g. topic) to be learned next in a Curriculum. Using the estimated affective state and cognitive state data of the subject, performance data of the subject, the Knowledge Tree (with all ‘edge’ topics listed), the compliance officer’s recommendation information, data from collaborative filters (look at data from peer subjects), and task performance specification content data (match subject attributes with the task performance specification material’s attributes), the recommendation engine recommends the next Task to be executed by the system under the Training Model. For example, the subject’s negative emotion can be eased by recognizing the difficult / unfamiliar topics (from the affective state data estimated during the execution of certain Task) and recommending the next Task of a different / more familiar topic; and recommending the next Task of a difficult / unfamiliar topic when subject’s emotion state is detected position. In another example, the recommendation engine can select the next Task of higher difficulty when the estimated affective state data shows that the subject is unchallenged. This allows the matching of Tasks with the highest compliance or learning/training level gains. This allows the clustering of Tasks based on similar performance data and/or affective state and cognitive state estimation. This also allows the matching of subject peers with similar compliance level accomplishment to form focus groups.
304. If the requested Task objects are found, their data are retrieved and are sent to the subject’s computing device for presentation in the system’s communication module user interface.
305. The subject selects a Task object to begin the compliance evaluation and monitoring session.
306. The system’s trainer module retrieves from the Domain Knowledge repository the next item in the selected Task object for rendering in the system’s communication module user interface.
307. Entering the Subject Model, the system’s communication module user interface renders the item (compliance questionnaire question and/or task procedure instruction) in the selected Task object.
308. A camera for capturing the subject’s face is activated.
309. During the subject’s engagement in task procedure materials in the item in the selected Task object (309a), the subject’s point-of-gaze and facial expressions are analyzed (309b).
310. Depending on the estimated affective state and cognitive state of the subject based on sensory data collected and information in the subject’s profile (overlay, includes all past performance data and compliance level achievement or lecture material understanding progress data), virtual assistant may be presented in the form of guidance and/or textual hint displayed in the system’s communication module user interface.
311. The subject submits an attempt answer and/or an attempt command for completing a task procedural step.
312. The attempt answer and/or attempt command is graded and the grade is displayed to the subject in the system’s communication module user interface.
313. The attempt answer and/or attempt command and grade is also stored by the system for further analysis.
314. The attempt answer and/or attempt command and grade are used in calculating the probability of the subject’s understanding of the Concept associated with the selected Task object and the probability of the subject achieving a target compliance or learning/training target in the task.
315. If the selected Task is completed, the system’s trainer module selects and requests the next Task based on the calculated probability of the subject’s understanding of the associated Concept and the probability of the subject achieving a target compliance or learning/training target in the task, and repeat the steps from step 303.
316. If the selected Task is not yet completed, the system’s trainer module retrieves the next item in the selected Task and repeat the steps from step 306.
317. After all Tasks are completed, the system generates the result report for subject.

In accordance to another aspect of the present invention, the system for administering, evaluating, and monitoring a subject’s compliance, adaptive and interactive learning, or neurocognitive disorder diagnosis with task performance requirements within an action programme further comprises an administration module that takes information from the compliance officers, subjects, and Domain Knowledge in offering assistance with the operation of face-to-face compliance evaluation and monitoring or in-class learning/training process across multiple physical facilities as well as online, remote evaluation and monitoring. In an exemplary embodiment, the administration module comprises a constraint-based scheduling algorithm that determines the optimal scheduling of compliance evaluation and monitoring sessions while observing constraints such compliance officers’ certification, travelling distance for subjects and compliance officers, first-come-first-served, composition of the compliance officers group based on compliance level achievement progress and training strategy. For example, when the compliance officer wants to promote peer teaching/training, the scheduling algorithm can select subjects with complementary skill sets so that they can help each other and form focus groups.

An in-person face-to-face compliance evaluation and monitoring session may comprise a typical flow such as: subjects check in, perform a small task to evaluate the cognitive state of the subjects, and the results are presented on the compliance officer’s (or the teacher/trainer’s or the medical service provider’s) user interface dashboard directly after completion. The session then continues with explanation of a new task performance specification by the compliance officer, teacher/trainer, or medical service provider, here the compliance officer, teacher/trainer, or medical service provider receives assistance from the system’s pedagogical agent with pedagogical goals and hints. After the explanation, the subjects may engage in the new task in which the system provides as much scaffolding as needed. Based on the compliance level achievement or learning/training progress and affective states of the subjects, the system’s trainer module decides how to continue the compliance evaluation and monitoring session with a few options: e.g., recommend to form focus groups each with subjects who have achieved similar compliance levels in prior tasks completed. The compliance evaluation and monitoring session, learning/training session, or neurocognitive disorder diagnosis is concluded by checking out. The attendance data is collected for billing purposes and for compliance certification purposes.

Although the embodiments of the present invention described above are primarily applied in commercial and industrial activities, surveying, and job performance assessment settings, the present invention can be adapted without undue experimentation to customer relationship management (CRM) action programmes. In accordance to one embodiment of the present invention, the method and system for administering, evaluating, and monitoring a subject’s compliance, interactive learning and training programmes, and neurocognitive disorder diagnosis with task performance requirements within an action programme comprise a mechanism for delivering and managing interactive and adaptive compliance questionnaire and task procedural instructions, the lecture materials, or the neurocognitive disorder diagnosis. The mechanism logically structures compliance questionnaire and task procedural instruction materials and the delivery mechanism data in a compliance programme as a Domain Knowledge, with its constituent Concept objects and Task objects having Knowledge and Skill items, and training materials respectively that are relevant to the concerned industry or trade. In the application of surveying, the system’s estimation of the subjects’ affective states and cognitive states can be used in driving the selection and presentment of survey questions. This in turn enables more accurate and speedy survey results procurements from the subjects. In the application of job performance assessment, the system’s estimation of the employee subjects’ affective states and cognitive states on duty continuously allows an employer to gauge the skill levels, engagement levels, and interests of the employees and in turn provides assistance in work and role assignments.

An in-class learning session may comprise a typical flow such as: subjects check in, perform a small quiz to evaluate the cognitive state of the subjects, and the results are presented on the teacher/trainer’s user interface dashboard directly after completion. The session then continues with class wide explanation of a new concept by the teacher/trainer, here the teacher/trainer receives assistance from the system’s pedagogical agent with pedagogical goals and hints. After the explanation, the subjects may engage with exercises/tasks in which the system provides as much scaffolding as needed. Based on the learning progress and affective states of the subjects, the system’s trainer module decides how to continue the learning session with a few options: e.g., provide educational games to deal with negative emotions, and allow two or more student subjects engage in a small competition for a small prize, digital badge, and the like. The learning session is concluded by checking out. The attendance data is collected for billing purposes and secondly for safety purposes as the parents can verify (or receive a notification from the system) of arrival and departure times of their children.

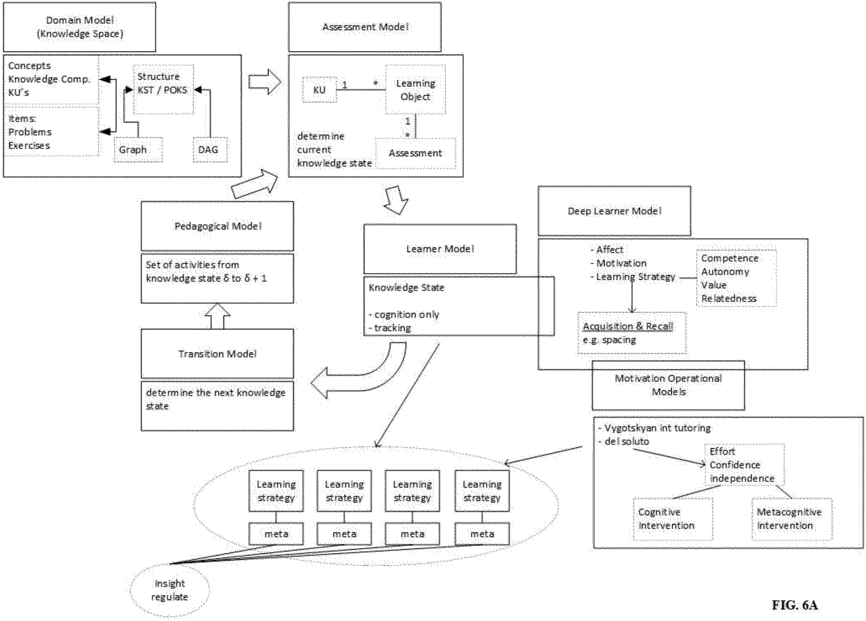

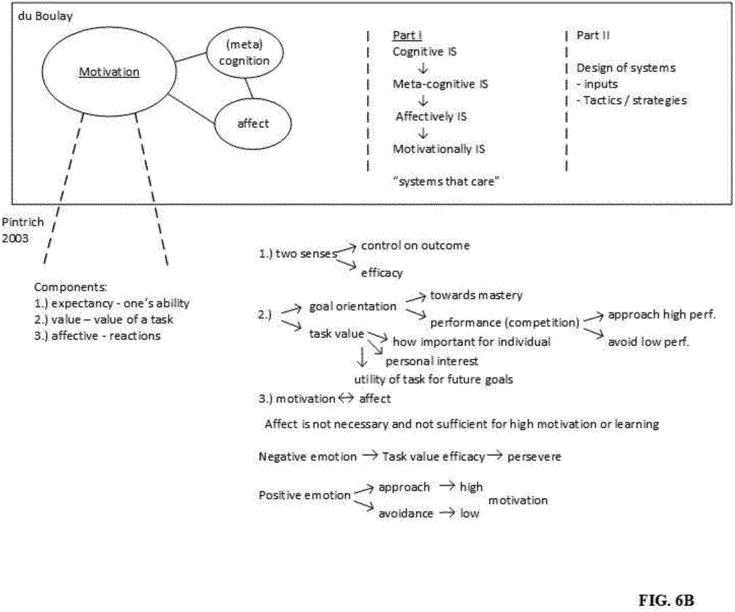

Referring to FIGS. 6A and 6B. In accordance to another aspect of the present invention, the method and system for method and system for administering, evaluating, and monitoring a subject’s compliance, interactive learning and training programmes, and neurocognitive disorder diagnosis with task performance requirements within an action programme incorporate machine learning techniques that are based on interlinking models of execution comprising: a Domain Model, an Assessment Model, a Learner Model, a Deep Learner Model, one or more Motivational Models, a Transition Model, and a Pedagogical Model. The interlinking models of execution is purposed for driving, inducing, or motivating certain desirable actions, behavior, and/or outcome from the subject. These certain desirable actions and/or outcome can be, as non-limiting examples, learning certain subject matters, achieving certain academic goals, achieving certain career goals, completing certain job assignments, making certain purchases, and conducting certain commercial activities. These interlinking models of execution together form a machine learning feedback loop comprising the continuous tracking and assessment of learning progress of the subject under the Assessment Model, driving the learning activities under the Learner Model, motivating the subject under the Deep Learner Model and Motivation Operational Model, and selecting and re-selecting knowledge space items under the Domain Model and Transition Model, and delivering the knowledge space items and activities from one knowledge state to the next under the Pedagogical Model.

In other words, one embodiment of the present invention provides a system for delivering and managing neurocognitive disorder diagnosis, comprising: one or more optical sensors configured for capturing and generating sensory data on a subject during a neurocognitive disorder diagnosis, wherein the sensory data comprises one or more of the subject’s pupillary responses, eye movements, point-of-gaze, facial expression, and head pose; one or more electronic databases including one or more domain knowledge data entities, each domain knowledge data entity comprising one or more concept data entities and neurocognitive disorder diagnosis test data entities, wherein each neurocognitive disorder diagnosis test data entity comprises one or more questionnaire and task procedural instruction material items, wherein each neurocognitive disorder diagnosis test data entity is associated with at least one concept data entity, and wherein a curriculum is formed by grouping a plurality of the concept data entities; a subject module executed by one or more computer processing devices configured to estimate the subject’s affective state and cognitive state using the sensory data collected from the optical sensors; a trainer module executed by one or more computer processing devices configured to select a subsequent neurocognitive disorder diagnosis test data entity and retrieve from the electronic databases the neurocognitive disorder diagnosis test data entity’s questionnaire for delivery and presentment to the subject after each completion of a neurocognitive disorder diagnosis test data entity in the neurocognitive disorder diagnosis; and a recommendation engine executed by one or more computer processing devices configured to create a list of neurocognitive disorder diagnosis test data entities available for selection of the subsequent neurocognitive disorder diagnosis test data entity, wherein the neurocognitive disorder diagnosis test data entities available for selection are the neurocognitive disorder diagnosis test data entities associated with the one or more concept data entities.

And another embodiment of the present invention providesA system for delivering and managing learning and training programmes, comprising: one or more optical sensors configured for capturing and generating sensory data on a subject during a learning or training session, wherein the sensory data comprises one or more of the subject’s pupillary responses, eye movements, point-of-gaze, facial expression, and head pose; one or more electronic databases including one or more domain knowledge data entities, each domain knowledge data entity comprising one or more concept data entities and one or more task data entities, wherein each concept data entity comprises one or more knowledge and skill content items, wherein each task data entity comprises one or more lecture content material items, wherein each task data entity is associated with at least one concept data entity, and wherein a curriculum is formed by grouping a plurality of the concept data entities; a subject module executed by one or more computer processing devices configured to estimate the subject’s affective state and cognitive state using the sensory data collected from the optical sensors; a trainer module executed by one or more computer processing devices configured to select a subsequent task data entity and retrieve from the electronic databases the task data entity’s lecture content material items for delivery and presentment to the subject after each completion of a task data entity in the learning or training session; and a recommendation engine executed by one or more computer processing devices configured to create a list of task data entities available for selection of the subsequent task data entity, wherein the task data entities available for selection are the task data entities associated with the one or more concept data entities forming the curriculum selected; wherein the selection of a task data entity from the list of task data entities available for selection is based on a probability of the subject achieving a target understanding of the concept data entity’s knowledge and skill content items; and wherein the probability of the subject achieving the target understanding is computed using input data of the estimation of the subject’s affective state and cognitive state.

The electronic embodiments disclosed herein may be implemented using general purpose or specialized computing devices, computer processors, or electronic circuitries including but not limited to application specific integrated circuits (ASIC), field programmable gate arrays (FPGA), and other programmable logic devices configured or programmed according to the teachings of the present disclosure. Computer instructions or software codes running in the general purpose or specialized computing devices, computer processors, or programmable logic devices can readily be prepared by practitioners skilled in the software or electronic art based on the teachings of the present disclosure. All or portions of the electronic embodiments may be executed in one or more general purpose or computing devices including server computers, personal computers, laptop computers, mobile computing devices such as smartphones and tablet computers. The electronic embodiments include computer storage media having computer instructions or software codes stored therein which can be used to program computers or microprocessors to perform any of the processes of the present invention. The storage media can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data. Various embodiments of the present invention also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention for various embodiments and with various modifications that are suited to the particular use contemplated.

What is claimed is:

1. A system for detection of noncompliance including substance abuse, driving under influence, and untruthful testimony giving, comprising:
one or more optical sensors configured for capturing and generating sensory data on a subject during a compliance evaluation and monitoring session, wherein the sensory data comprises one or more of the subject’s pupillary responses, eye movements, point-of-gaze, facial expression, and head pose;
one or more electronic databases including one or more domain knowledge data entities, each domain knowledge data entity comprising one or more concept data entities and one or more task data entities, wherein each concept data entity comprises one or more and task performance specification content items, wherein each task data entity comprises one or more compliance questionnaire and task procedural instruction material items, wherein each task data entity is associated with at least one concept data entity, and wherein a curriculum is formed by grouping a plurality of the concept data entities;
a subject module executed by one or more computer processing devices configured to estimate the subject’s affective state and cognitive state using the sensory data collected from the optical sensors;
a trainer module executed by one or more computer processing devices configured to select a subsequent task data entity and retrieve from the electronic databases the task data entity’s compliance questionnaire and task procedural instruction material items for delivery and presentment to the subject after each completion of a task data entity in the compliance evaluation and monitoring session; and
a recommendation engine executed by one or more computer processing devices configured to create a list of task data entities available for selection of the subsequent task data entity, wherein the task data entities available for selection are the task data entities associated with the one or more concept data entities forming the curriculum selected;
wherein the selection of a task data entity from the list of task data entities available for selection is based on a probability of the subject achieving a target compliance level in a task associated with the concept data entity’s task performance specification content items; and
wherein the probability of the subject achieving the target compliance level is computed using input data of the estimation of the subject’s affective state and cognitive state.

2. The system of claim 1, further comprising:
one or more physiologic measuring devices configured for capturing one or more of the subject’s tactile pressure exerted on a tactile sensing device, heart rate, electro dermal activity (EDA), skin temperature, and touch response, and generating additional sensory data during the compliance evaluation and monitoring session;
wherein the subject module is further configured to estimate the subject’s affective state and cognitive state using the sensory data collected from the optical sensors and the additional sensory data collected from the physiologic measuring devices.

3. The system of claim 1, further comprising:
one or more voice recording devices configured for capturing the subject’s voice and speech clarity, and generating additional sensory data during the compliance evaluation and monitoring session;
wherein the subject module is further configured to estimate the subject’s affective state and cognitive state using the sensory data collected from the optical sensors and the additional sensory data collected from the voice recording devices.

4. The system of claim 1, further comprising:
one or more handwriting capturing devices configured for capturing the subject’s handwriting, and generating additional sensory data during the compliance evaluation and monitoring session;
wherein the module is further configured to estimate the subject’s affective state and cognitive state using the sensory data collected from the optical sensors and the additional sensory data collected from the handwriting capturing devices.

5. The system of claim 1, further comprising:
one or more pedagogical agents configured for capturing the subject’s interaction with the pedagogical agents, and generating additional sensory data during the compliance evaluation and monitoring session;
wherein the module is further configured to estimate the subject’s affective state and cognitive state using the sensory data collected from the optical sensors and the additional sensory data collected from the pedagogical agents.

6. The system of claim 1, wherein each of the task performance specification content material items is an operation note, an illustration, a test question, a video with an embedded test question, a problem-solving exercise having multiple steps designed to provide guidance in deriving a solution to a problem, or a problem-solving exercise having one or more heuristic rules or constraints for simulating problem-solving exercise steps delivered in synchronous with the subject’s performance of the task procedural steps associated with the task performance specification.

7. The system of claim 1,
wherein a plurality of the concept data entities are linked to form a logical tree data structure;
wherein concept data entities having knowledge and skill content items that are fundamental in a topic are represented by nodes closer to a root of the logical tree data structure and concept data entities having knowledge and skill content items that are advance and branches of a common fundamental knowledge and skill content item are represented by nodes higher up in different branches of the logical tree data structure;
wherein the recommendation engine is further configured to create a list of task data entities available for selection of the subsequent task data entity, wherein the task data entities available for selection are the task data entities associated with the one or more concept data entities forming the curriculum selected and the one or more concept data entities having knowledge and skill items not yet mastered by the subject and as close to the roots of the logical tree data structures that the concept data entities belonging to.

8. The system of claim 1,
wherein the probability of the subject achieving the target compliance level is computed using input data of the estimation the subject’s affective state and cognitive state and the subject’s performance data and behavioral data; and
wherein the subject’s performance data and behavioral data comprises one or more of number of successful and unsuccessful attempts to task procedural step completions, speed in completing task procedures, correctness of answers to questions in the questionnaire, number of successful and unsuccessful attempts to questions, closeness of the subject’s answers to model answers, toggling between given answer choices, and response speed to test questions of certain types, subject matters, and/or task performance specification complexity/difficulty/stringency levels, working steps toward a solution, the subject’s handwriting, tone of voice, and speech clarity.

9. The system of claim 1,
wherein the selection of a task data entity from the list of task data entities available for selection is based on the probability of the subject achieving the target compliance level and the subject’s estimated affective state;
wherein when the subject’s estimated affective state indicates a negative emotion, a task data entity that is associated with a concept data entity having knowledge and skill content items that are favored by the subject is selected over another task data entity that is associated with another concept data entity having knowledge and skill content items that are disliked by the subject; and
wherein when the subject’s estimated affective state indicates a positive emotion, a task data entity that is associated with a concept data entity having knowledge and skill content items that are disliked by the subject is selected over another task data entity that is associated with another concept data entity having knowledge and skill content items that are favored by the subject.

10. A system for delivering and managing neurocognitive disorder diagnosis, comprising:
one or more optical sensors configured for capturing and generating sensory data on a subject during a neurocognitive disorder diagnosis, wherein the sensory data comprises one or more of the subject’s pupillary responses, eye movements, point-of-gaze, facial expression, and head pose;
one or more electronic databases including one or more domain knowledge data entities, each domain knowledge data entity comprising one or more concept data entities and neurocognitive disorder diagnosis test data entities, wherein each neurocognitive disorder diagnosis test data entity comprises one or more questionnaire and task procedural instruction material items, wherein each neurocognitive disorder diagnosis test data entity is associated with at least one concept data entity, and wherein a curriculum is formed by grouping a plurality of the concept data entities;
a subject module executed by one or more computer processing devices configured to estimate the subject’s affective state and cognitive state using the sensory data collected from the optical sensors;
a trainer module executed by one or more computer processing devices configured to select a subsequent neurocognitive disorder diagnosis test data entity and retrieve from the electronic databases the neurocognitive disorder diagnosis test data entity’s questionnaire for delivery and presentment to the subject after each completion of a neurocognitive disorder diagnosis test data entity in the neurocognitive disorder diagnosis; and
a recommendation engine executed by one or more computer processing devices configured to create a list of neurocognitive disorder diagnosis test data entities available for selection of the subsequent neurocognitive disorder diagnosis test data entity, wherein the neurocognitive disorder diagnosis test data entities available for selection are the neurocognitive disorder diagnosis test data entities associated with the one or more concept data entities.

11. The system of claim 10, further comprising:
one or more physiologic measuring devices configured for capturing one or more of the subject’s tactile pressure exerted on a tactile sensing device, heart rate, electro dermal activity (EDA), skin temperature, and touch response, and generating additional sensory data during the neurocognitive disorder diagnosis;
wherein the subject module is further configured to estimate the subject’s affective state and cognitive state using the sensory data collected from the optical sensors and the additional sensory data collected from the physiologic measuring devices.

12. The system of claim 10, further comprising:
one or more voice recording devices configured for capturing the subject’s voice and speech clarity, and generating additional sensory data during the neurocognitive disorder diagnosis;
wherein the subject module is further configured to estimate the subject’s affective state and cognitive state using the sensory data collected from the optical sensors and the additional sensory data collected from the voice recording devices.

13. The system of claim 10, further comprising:
one or more handwriting capturing devices configured for capturing the subject’s handwriting, and generating additional sensory data during the neurocognitive disorder diagnosis;
wherein the module is further configured to estimate the subject’s affective state and cognitive state using the sensory data collected from the optical sensors and the additional sensory data collected from the handwriting capturing devices.

14. The system of claim 10, further comprising:
one or more pedagogical agents configured for capturing the subject’s interaction with the pedagogical agents, and generating additional sensory data during the neurocognitive disorder diagnosis;
wherein the module is further configured to estimate the subject’s affective state and cognitive state using the sensory data collected from the optical sensors and the additional sensory data collected from the pedagogical agents.

15. A system for delivering and managing learning and training programmes, comprising:
one or more optical sensors configured for capturing and generating sensory data on a subject during a learning or training session, wherein the sensory data comprises one or more of the subject’s pupillary responses, eye movements, point-of-gaze, facial expression, and head pose;
one or more electronic databases including one or more domain knowledge data entities, each domain knowledge data entity comprising one or more concept data entities and one or more task data entities, wherein each concept data entity comprises one or more knowledge and skill content items, wherein each task data entity comprises one or more lecture content material items, wherein each task data entity is associated with at least one concept data entity, and wherein a curriculum is formed by grouping a plurality of the concept data entities;
a subject module executed by one or more computer processing devices configured to estimate the subject’s affective state and cognitive state using the sensory data collected from the optical sensors;
a trainer module executed by one or more computer processing devices configured to select a subsequent task data entity and retrieve from the electronic databases the task data entity’s lecture content material items for delivery and presentment to the subject after each completion of a task data entity in the learning or training session; and
a recommendation engine executed by one or more computer processing devices configured to create a list of task data entities available for selection of the subsequent task data entity, wherein the task data entities available for selection are the task data entities associated with the one or more concept data entities forming the curriculum selected;
wherein the selection of a task data entity from the list of task data entities available for selection is based on a probability of the subject achieving a target understanding of the concept data entity’s knowledge and skill content items; and
wherein the probability of the subject achieving the target understanding is computed using input data of the estimation of the subject’s affective state and cognitive state.

16. The system of claim 15, further comprising:
one or more physiologic measuring devices configured for capturing one or more of the subject’s tactile pressure exerted on a tactile sensing device, heart rate, electro dermal activity (EDA), skin temperature, and touch response, and generating additional sensory data during the learning or training session;
wherein the subject module is further configured to estimate the subject’s affective state and cognitive state using the sensory data collected from the optical sensors and the additional sensory data collected from the physiologic measuring devices.

17. The system of claim 15, further comprising:
one or more voice recording devices configured for capturing the subject’s voice and speech clarity, and generating additional sensory data during the learning or training session;
wherein the subject module is further configured to estimate the subject’s affective state and cognitive state using the sensory data collected from the optical sensors and the additional sensory data collected from the voice recording devices.

18. The system of claim 15, further comprising:
one or more handwriting capturing devices configured for capturing the subject’s handwriting, and generating additional sensory data during the learning or training session;
wherein the module is further configured to estimate the subject’s affective state and cognitive state using the sensory data collected from the optical sensors and the additional sensory data collected from the handwriting capturing devices.

19. The system of claim 15, further comprising:
one or more pedagogical agents configured for capturing the subject’s interaction with the pedagogical agents, and generating additional sensory data during the learning or training session;
wherein the module is further configured to estimate the subject’s affective state and cognitive state using the sensory data collected from the optical sensors and the additional sensory data collected from the pedagogical agents.
